(19) United States
(12) Patent Application Publication
(10) Pub. No.: US 2019/0231272 A1
(43) Pub. Date: Aug. 1, 2019

(54) MEASURING APPARATUS, MEASURING METHOD, AND NON-TRANSITORY COMPUTER-READABLE STORAGE MEDIUM FOR STORING PROGRAM

(71) Applicant: FUJITSU LIMITED, Kawasaki-shi (JP)

(72) Inventor: TAKAYUKI YAMAJI, Yokohama (JP)

(73) Assignee: FUJITSU LIMITED, Kawasaki-shi (JP)

(21) Appl. No.: 16/376,457

(22) Filed: Apr. 5, 2019

Related U.S. Application Data
(63) Continuation of application No. PCT/JP2016/081138, filed on Oct. 20, 2016.

(51) Int. Cl. A61B 5/00; G01S 13/88; G01S 7/41; A61B 5/11; A61B 5/0205

(52) U.S. Cl. CPC A61B 5/6893; G01S 13/88; G01S 7/415; A61B 5/024; A61B 5/0205; A61B 2562/0219; A61B 5/1126

(57) ABSTRACT

A measuring method includes: executing first acquisition processing for acquiring a first signal from a first sensor, the first sensor being provided at a first position corresponding to a height of a waist of an occupant in a backrest of a seat on which the occupant sits, the first sensor being configured to detect a movement in accordance with first radio waves; executing second acquisition processing for acquiring a second signal from a second sensor, the second sensor being provided at a second position corresponding to a height of a chest of the occupant in the backrest, the second sensor being configured to detect a movement in accordance with second radio waves; executing generation processing for generating a measurement result related to a predetermined component included in the second signal based on the first signal and the second signal; and executing output processing for outputting the generated measurement result.

### CROSS-REFERENCE TO RELATED APPLICATION

This application is a continuation application of International Application PCT/JP2016/081138 filed on Oct. 20, 2016 and designated the U.S., the entire contents of which are incorporated herein by reference.

### FIELD

The embodiments discussed herein are related to a measuring apparatus, a measuring method, and a non-transitory computer-readable storage medium for storing a program.

### BACKGROUND

In the related art, there has been known a technique of accurately measuring information of an occupant under an environment such as an inside of a vehicle.

Examples of the related art include International Publication Pamphlet No. WO 2010/107091, International Publication Pamphlet No. WO 2010/107093, and Japanese Laid-open Patent Publication No. 2011-30869.

### SUMMARY

According to an aspect of the embodiments, a measuring method performed by a computer includes: executing first acquisition processing that includes acquiring a first signal from a first sensor, the first sensor being provided at a first position corresponding to a height of a waist of an occupant in a backrest of a seat on which the occupant sits, the first sensor being configured to detect a movement in accordance with first radio waves; executing second acquisition processing that includes acquiring a second signal from a second sensor, the second sensor being provided at a second position corresponding to a height of a chest of the occupant in the backrest, the second sensor being configured to detect a movement in accordance with second radio waves; executing generation processing that includes generating a measurement result related to a predetermined component included in the second signal based on the first signal and the second signal; and executing output processing that includes outputting the generated measurement result.

The object and advantages of the invention will be realized and attained by means of the elements and combinations particularly pointed out in the claims.

It is to be understood that both the foregoing general description and the following detailed description are exemplary and explanatory and are not restrictive of the invention.

### BRIEF DESCRIPTION OF DRAWINGS

FIG. 1A is a view illustrating an example of a mounting state of Doppler sensor;

FIG. 1B is a plan view schematically illustrating a backrest on which the Doppler sensor is mounted;

FIG. 2 is an explanatory view of a configuration of one Doppler sensor;

FIG. 3 is a block diagram illustrating an example of a hardware configuration of a measuring apparatus according to Example 1;

FIG. 4 is a block diagram illustrating an example of one radio wave transmitting and receiving unit;

FIG. 5 is a block diagram illustrating an example of a functional configuration of the measuring apparatus according to Example 1;

FIG. 6 is a graph illustrating an example of an analysis result obtained by a frequency analysis for a waist sensor signal;

FIG. 7 is a graph illustrating an example of a chest sensor signal;

FIG. 8 is a graph illustrating an example of an analysis result obtained by a frequency analysis for the chest sensor signal;

FIG. 9 is a graph illustrating an example of a heartbeat filtering signal;

FIG. 10 is a graph illustrating an output example of a heartbeat interval;

FIG. 11 is a flowchart illustrating an operation example of the measuring apparatus;

FIG. 12 is a view illustrating an example of a mounting state of an inertial sensor;

FIG. 13 is a block diagram illustrating an example of a functional configuration of a measuring apparatus according to Example 2;

FIG. 14 is a graph illustrating an example of an analysis result obtained by a frequency analysis for an inertial sensor signal;

FIG. 15 is an explanatory view of an effect of Example 2; and

FIG. 16 is a flowchart illustrating an operation example of the measuring apparatus.

### DESCRIPTION OF EMBODIMENTS

However, the technique of the related art described above is difficult to be applied to realize accurate measurement using radio waves. For example, when an occupant exists under an environment where vibration is likely to occur, it is difficult to increase the accuracy of a measurement result related to a desired component due to a component (noise component) related to the vibration that may be included in a sensor signal.

Therefore, in one aspect, an embodiment aims to realize accurate measurement using radio waves.

Hereinafter, each example will be described with reference to the drawings in detail.

In the following examples, a measuring apparatus measures information (information related to heartbeat or breathing of an occupant) of the occupant using a sensor that uses the radio waves for measurement such as Doppler sensor.

First, prior to the explanation of the measuring apparatus, the Doppler sensor will be described.

The Doppler sensor may irradiate an object with the radio waves such as microwaves and capture a movement of the object from an amount of a change of reflected waves from the object. As a basic principle, when Doppler effect is used and a distance to the object irradiated and reflected with the radio waves is changed, a reflection amount is changed, so that the reflection amount is captured as a signal by being converted into a voltage value. When the radio waves are applied to a living body, various pieces of information are included in a signal reflected from the living body due to a body surface, a heartbeat, breathing, a body movement, or the like.

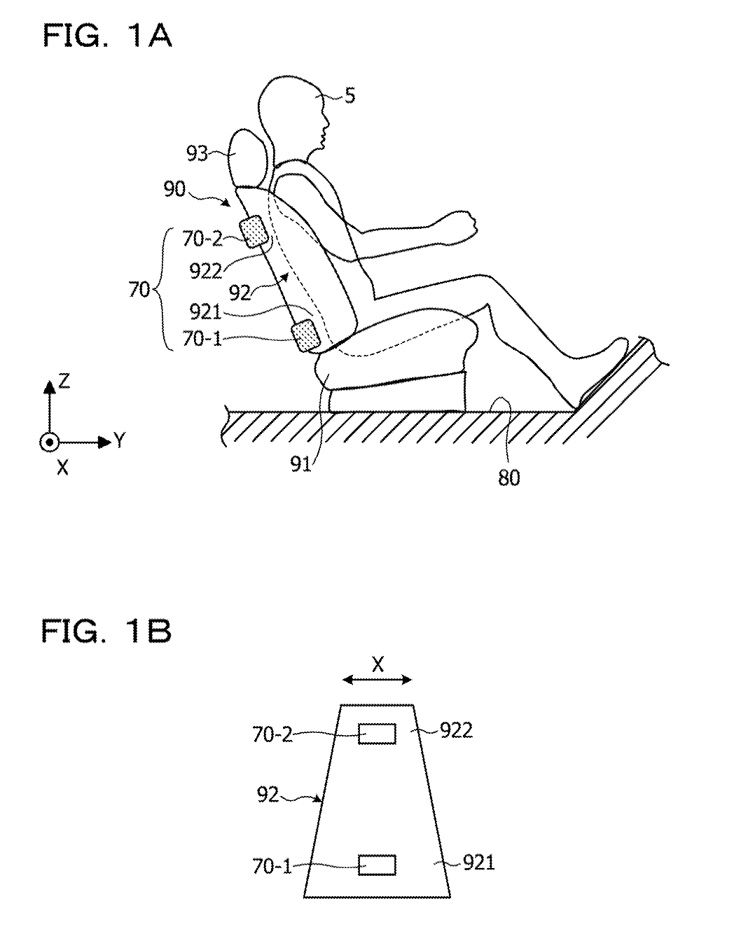

FIG. 1A is a view illustrating an example of a mounting state of Doppler sensor 70. FIG. 1A schematically illustrates an occupant 5 sitting (seating) on a seat 90 as a side view. The Doppler sensor 70 provided in the seat 90 is schematically illustrated in FIG. 1A as a perspective view. FIG. 1A (same applies to FIG. 1B described later) is provided for explaining a mounting position of the Doppler sensor 70 and schematically illustrates the Doppler sensor 70. FIG. 1A illustrates respective X-, Y-, and Z-axes as three axes orthogonal to each other. In a stationary state (stationary state on land in a case of an aircraft) of a vehicle, an XY plane corresponds to a horizontal plane and the Z-axis corresponds to a vertical direction (gravity direction). FIG. 1B is a plan view schematically illustrating a backrest on which the Doppler sensor 70 is mounted.

The seat 90 is attached to the vehicle. The vehicle is a motorcycle, an automobile (four wheels), a truck, a bus, a ship, an aircraft, a construction machinery, or the like. The occupant 5 is an occupant or a driver of the vehicle, but may be a passenger other than the driver. A space (cabin, cockpit, or the like) of the vehicle is an example of an environment where noise due to vibration or the like is likely to occur. In the example, as will be described later, accurate measurement may be realized even in the environment where noise is likely to occur, so that it is preferred that the vehicle is, for example, a vehicle with a relatively intense movement (such as generation of a relatively high acceleration) such as a training aircraft. The example is suitable for a vehicle in which vibration in a use state occurs in various modes and the vibration is relatively large.

The seat 90 may be directly fixed to a vehicle floor 80 or may be slidably attached to the vehicle floor 80 via a sliding mechanism (not illustrated). In a state where the occupant 5 is seating on the seat 90, the occupant 5 may be restrained by a seat belt (not illustrated) or the like to the seat 90.

The seat 90 includes a seat surface portion 91, a backrest 92, and a headrest 93. A material of the seat 90 is arbitrary and a surface layer may be a fiber or a skin. The seat 90 may be a type in which an angle of the backrest 92 with respect to the seat surface portion 91 is variable (for example, a type in which reclining may be performed) or may be a type in which reclining may not be performed. The seat 90 may include a damper (vibration damping rubber or spring) so as not to directly transmit an input such as vibration transmitted to the vehicle floor 80 to the occupant 5.

The backrest 92 includes a lower portion 921 and an upper portion 922. The lower portion 921 rises from the seat surface portion 91. The lower portion 921 includes a portion that hits the waist (behind the lumbar vertebra) of the seated occupant 5 and mainly supports the waist of the occupant 5. The upper portion 922 includes a portion that hits above the waist of a back of the seated occupant 5 and mainly supports an upper side (behind the thoracic vertebra) of the back of the occupant 5.

Two Doppler sensors 70 are provided on the backrest 92. The Doppler sensor 70 may be built inside a surface of the backrest 92. Hereinafter, when the two Doppler sensors 70 are distinguished, they are referred to as Doppler sensors 70-1 and 70-2.

The Doppler sensor 70-1 (example of a first sensor) is provided in the lower portion 921 of the backrest 92. For example, as illustrated in FIG. 1A, the Doppler sensor 70-1 is provided at a position (example of a first position) corresponding to a height of the waist of the occupant 5. Functionally, the Doppler sensor 70-1 is provided in the lower portion 921 so that radio waves may be transmitted in a direction in which the waist of the occupant 5 is located. Therefore, the Doppler sensor 70-1 may detect the movement of the waist of the occupant 5 based on reflected waves of the transmitted radio waves.

As illustrated in FIG. 1B, the Doppler sensor 70-1 is preferably provided at a center portion of the lower portion 921 of the backrest 92 in a width direction (X direction in FIG. 1B). Therefore, there is a high possibility that the radio waves striking the waist of the occupant 5 seated on the seat 90 may be transmitted. An emission direction of the radio waves by the Doppler sensor 70-1 is substantially perpendicular to a surface of the lower portion 921 at a mounting location.

The Doppler sensor 70-2 (example of a second sensor) is provided in the upper portion 922 of the backrest 92. For example, as illustrated in FIG. 1A, the Doppler sensor 70-2 is provided at a position (example of a second position) corresponding to a height of a chest of the occupant 5. Functionally, the Doppler sensor 70-2 is provided in the upper portion 922 so that radio waves may be transmitted in a direction in which the chest of the occupant 5 is located. Therefore, the Doppler sensor 70-2 may detect a movement of the chest (including the lung, the heart, or the like) of the occupant 5 based on the reflected waves of the transmitted radio waves.

As illustrated in FIG. 1, the Doppler sensor 70-2 is preferably provided at a center portion of the upper portion 922 of the backrest 92 in the width direction (X direction in FIG. 1B). Therefore, there is a high possibility that the radio waves striking the chest of the occupant 5 seated on the seat 90 may be transmitted. An emission direction of the radio waves by the Doppler sensor 70-2 is substantially perpendicular to a surface of the upper portion 922 at a mounting location.

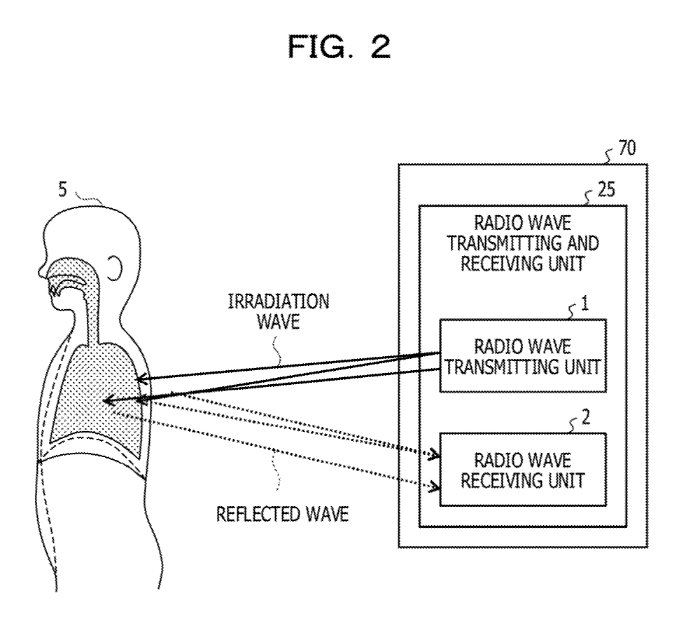

FIG. 2 is an explanatory view of a configuration of one Doppler sensor 70. FIG. 2 also illustrates the occupant 5 together with one Doppler sensor 70 for the purpose of explanation.

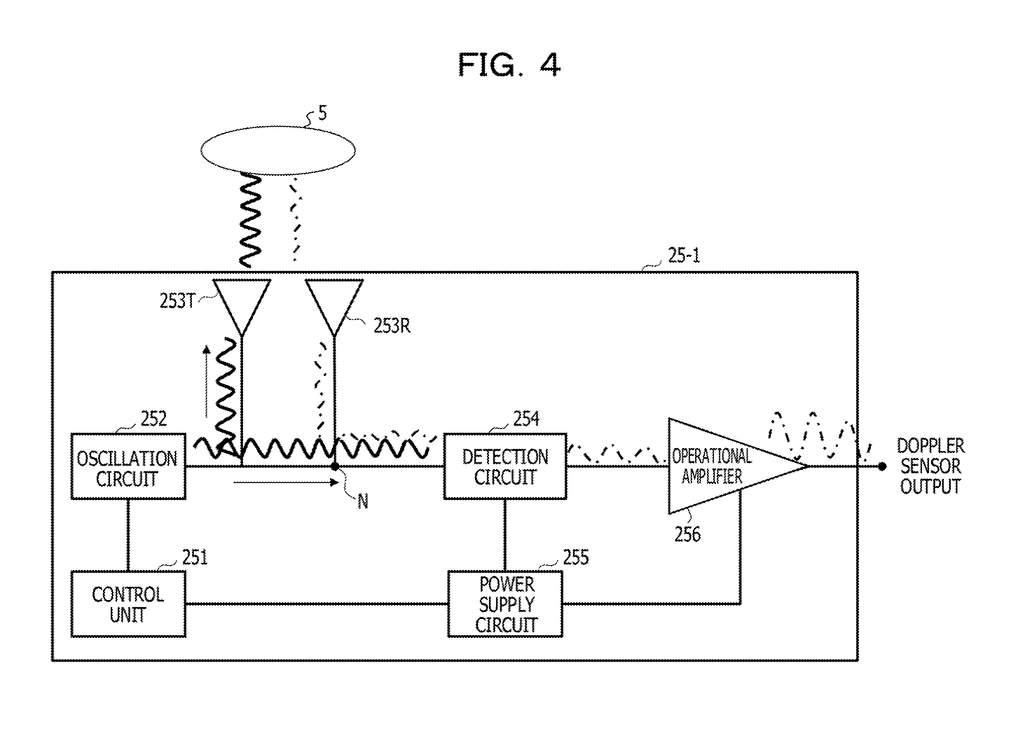

The Doppler sensor 70 includes a radio wave transmitting and receiving unit 25 and the radio wave transmitting and receiving unit 25 includes a radio wave transmitting unit 1 and a radio wave receiving unit 2. Details of the radio wave transmitting and receiving unit 25 will be described later with reference to FIG. 4.

The radio wave transmitting unit 1 irradiates a human body of the occupant 5 with the radio waves. A band of the radio waves is arbitrary. An ultra high frequency (UHF) and a super high frequency (SHF) are examples of the radio waves. The radio waves may be, for example, in 2.4 G band. The radio wave receiving unit 2 receives the reflected waves of the radio waves from the occupant 5.

The Doppler sensor 70 illustrated in FIG. 2 corresponds to the Doppler sensor 70-2 in a positional relationship with the occupant 5, and the same is applied to the configuration itself of the Doppler sensor 70-1. Hereinafter, the radio wave transmitting and receiving unit 25 of the Doppler sensor 70-1 will be referred to as a radio wave transmitting and receiving unit 25-1 and the radio wave transmitting and receiving unit 25 of the Doppler sensor 70-2 will be referred to as a radio wave transmitting and receiving unit 25-2.

Next, several examples of the measuring apparatus for executing a measurement using the Doppler sensors 70-1 and 70-2 will be described in order.

### Example 1

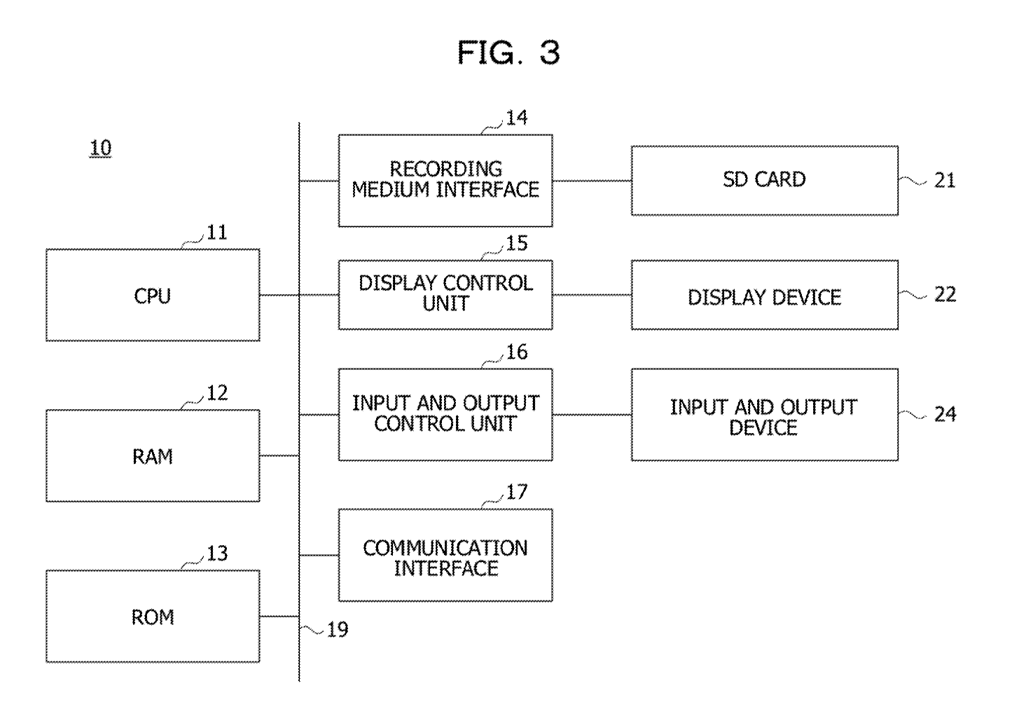

FIG. 3 is a block diagram illustrating an example of a hardware configuration of a measuring apparatus 10 according to Example 1. The measuring apparatus 10 forms an example of a measuring system by combining with the Doppler sensors 70-1 and 70-2.

The measuring apparatus 10 is formed by a computer (example of a processing device). In the example illustrated in FIG. 3, the measuring apparatus 10 includes a central processing unit (CPU) 11, a random access memory (RAM) 12, a read only memory (ROM) 13, a recording medium interface 14, and a display control unit 15 which are connected via a bus 19. The measuring apparatus 10 includes an input and output control unit 16 and a communication interface 17. A recording medium such as a secure digital (SD) card (or a memory card) 21 may be connected to the recording medium interface 14. A display device 22 is connected to the display control unit 15. An input and output device 24 is connected to the input and output control unit 16. The input and output device 24 may be a touch panel, a speaker, or the like. Functions of the display device 22 and the input and output device 24 may be realized by the touch panel. The recording medium interface 14 and the SD card 21, the input and output control unit 16 and the input and output device 24, the display device 22 and the display control unit 15, and/or a wireless transmitting and receiving unit 26 may be appropriately omitted.

The communication interface 17 is an interface for performing wired or wireless communication with an outside. The wireless communication may be realized via a wireless communication network in a mobile phone, a near field communication (NFC), a Bluetooth (registered trademark), a Wireless-Fidelity (Wi-Fi), Infrared, or the like. The Doppler sensors 70-1 and 70-2 may be connected to the communication interface 17. The measuring apparatus 10 acquires sensor signals, which are described later, from the Doppler sensors 70-1 and 70-2 via the communication interface 17.

The CPU 11 has a function of controlling an entire operation of the measuring apparatus 10. The RAM 12 and the ROM 13 form storage units for storing a program executed by the CPU 11 and various data. The program includes a program causing the CPU 11 to execute measurement processing and to function as the measuring apparatus. The storage unit may include the SD card 21. The storage unit for storing the program is an example of a computer readable storage medium.

The display device 22 has a function of displaying a result of the measurement processing or the like under the control of the display control unit 15.

FIG. 4 is a block diagram illustrating an example of the radio wave transmitting and receiving unit 25-1. The same may be applied to the radio wave transmitting and receiving unit 25-2.

The radio wave transmitting and receiving unit 25-1 includes a control unit 251, an oscillation circuit 252, antennas 253T and 253R, a detection circuit 254, a power supply circuit 255, and operational amplifiers 256 and 258. A transmission wave (radio wave) generated by the oscillation circuit 252 is branched into the antenna 253T and the detection circuit 254, and the occupant 5 is irradiated with a transmission wave transmitted from the antenna 253T. The transmission wave with which the occupant 5 is irradiated is reflected and a reflected wave of the transmission wave from the occupant 5 is received by the antenna 253R. The reflected wave, which is received by the antenna 253R and illustrated by a one-dot chain line, interferes with the transmission wave illustrated by a solid line in a node N, and a composite wave (DC component) illustrated by a one-dot chain line is output from the detection circuit 254. The operational amplifier 256 outputs a sensor output obtained by amplifying the composite wave via the communication interface 17. The sensor output (see Doppler sensor output of FIG. 4) from the operational amplifier 256 is also referred to as a sensor signal.

The power supply circuit 255 includes a battery that supplies a power supply voltage to the control unit 251, the oscillation circuit 252, the detection circuit 254, and the operational amplifier 256. The battery is, for example, a rechargeable battery. It goes without saying that the power supply circuit 255 may be externally connected to the radio wave transmitting and receiving unit 25-1. The antennas 253T and 253R may be integrated as transmitting and receiving antennas.

In the example of FIG. 4, the radio wave transmitting unit 1 includes at least the oscillation circuit 252 and the antenna 253T, and the radio wave receiving unit 2 includes at least the antenna 253R, the detection circuit 254, and the operational amplifier 256.

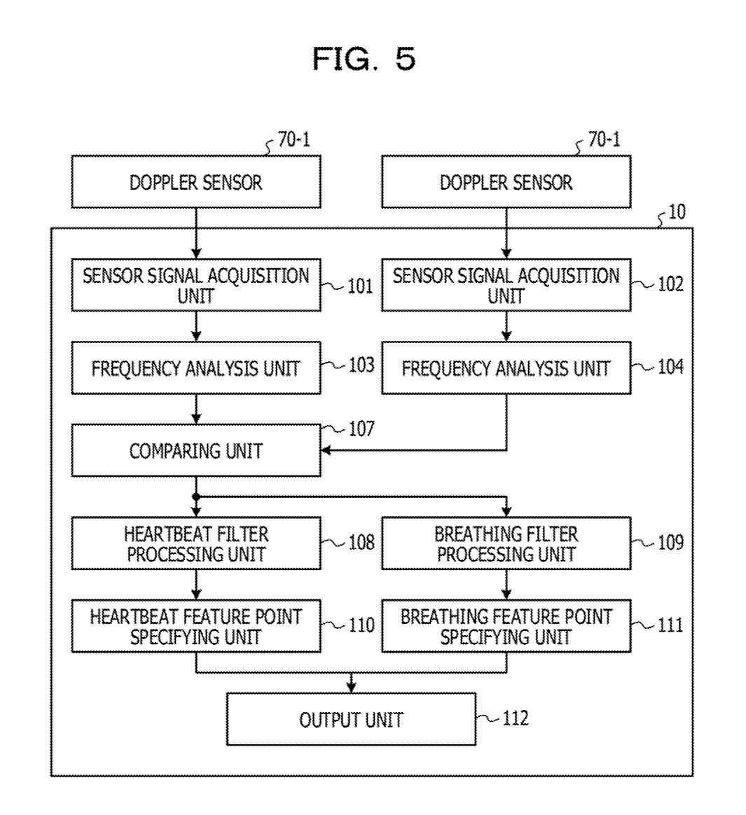

FIG. 5 is a block diagram illustrating an example of a functional configuration of the measuring apparatus 10 according to Example 1. FIG. 5 also illustrates the Doppler sensors 70-1 and 70-2.

The measuring apparatus 10 includes sensor signal acquisition units 101 and 102, and frequency analysis units 103 and 104. The sensor signal acquisition unit 101 and the frequency analysis unit 103 are a system related to the Doppler sensor 70-1, and the sensor signal acquisition unit 102 and the frequency analysis unit 104 are a system related to the Doppler sensor 70-2. The sensor signal acquisition units 101 and 102, and the frequency analysis units 103 and 104 may be realized by executing one or more programs stored in the ROM 13 by the CPU 11 illustrated in FIG. 4.

The measuring apparatus 10 further includes a comparing unit 107, a heartbeat filter processing unit 108, a breathing filter processing unit 109, a heartbeat feature point specifying unit 110, a breathing feature point specifying unit 111, and an output unit 112. The comparing unit 107, the heartbeat filter processing unit 108, the breathing filter processing unit 109, the heartbeat feature point specifying unit 110, the breathing feature point specifying unit 111, and the output unit 112 may be realized by executing one or more programs stored in the ROM 13 by the CPU 11 illustrated in FIG. 4.

The sensor signal acquisition unit 101 acquires (receives) a sensor signal (see Doppler sensor output in FIG. 4) from the radio wave transmitting and receiving unit 25-1 of the Doppler sensor 70-1. Hereinafter, the sensor signal (example of a first signal) from the radio wave transmitting and receiving unit 25-1 is also referred to as a “waist sensor signal”. Hereinafter, the waist sensor signal is obtained in a state where the occupant 5 seated on the seat 90.

The sensor signal acquisition unit 102 acquires (receives) a sensor signal from the radio wave transmitting and receiving unit 25-2 of the Doppler sensor 70-2. Hereinafter, the sensor signal (example of a second signal) from the radio wave transmitting and receiving unit 25-2 is also referred to as a “chest sensor signal”. Hereinafter, the chest sensor signal is obtained in a state where the occupant 5 seated on the seat 90.

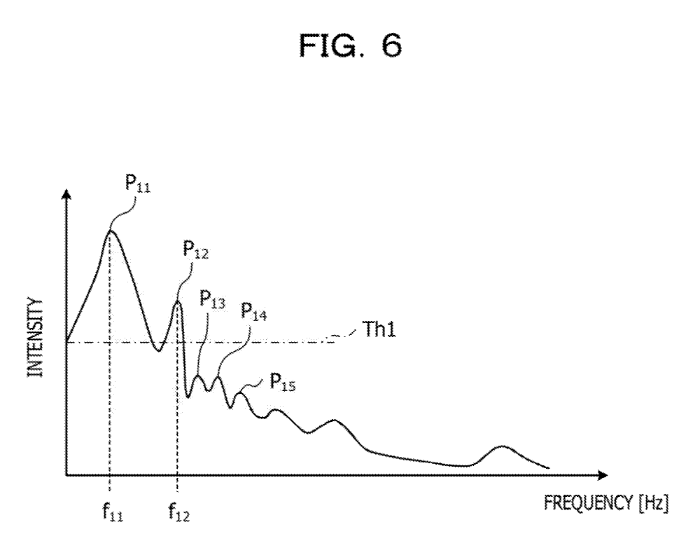

The frequency analysis unit 103 specifies a frequency at which a feature point is obtained based on the waist sensor signal. Specifically, the frequency analysis unit 103 acquires a power spectrum by performing a frequency analysis for the waist sensor signal obtained from the sensor signal acquisition unit 101. The frequency analysis is, for example, a Fast Fourier Transform (FFT) and, for example, an analysis result illustrated in FIG. 6 is obtained. FIG. 6 is a graph illustrating an example of the analysis result obtained by the frequency analysis for the waist sensor signal. In FIG. 6, a horizontal axis represents a frequency and a vertical axis represents intensity (power).

The frequency analysis unit 103 specifies a frequency at which peaks having a predetermined threshold Th1 or more are generated from the analysis result as illustrated in FIG. 6 as a frequency (hereinafter, referred to as a “first peak frequency”) at which the feature point is obtained. The predetermined threshold Th1 is a threshold for extracting only a frequency at which a significant feature point is obtained, and is an adaptive value. A plurality of first peak frequencies may exist. FIG. 6 illustrates peaks P₁₁ and P₁₂ as examples of peaks of the predetermined threshold Th1 or more. The peaks P₁₁ and P₁₂ (same is applied to other peaks P₁₃, P₁₄, and P₁₅ less than the predetermined threshold Th1) are peaks due to a noise component as will be described later. For example, the peaks P₁₁ and P₁₂ are feature points generated due to the movement (movement of the waist) of the occupant 5 with respect to the seat 90. The movement of the occupant 5 with respect to the seat 90 causing such peaks P₁₁ and P₁₂ to be generated is generated due to the movement and vibration of the vehicle itself, the movement of the occupant 5 himself or herself, or the like.

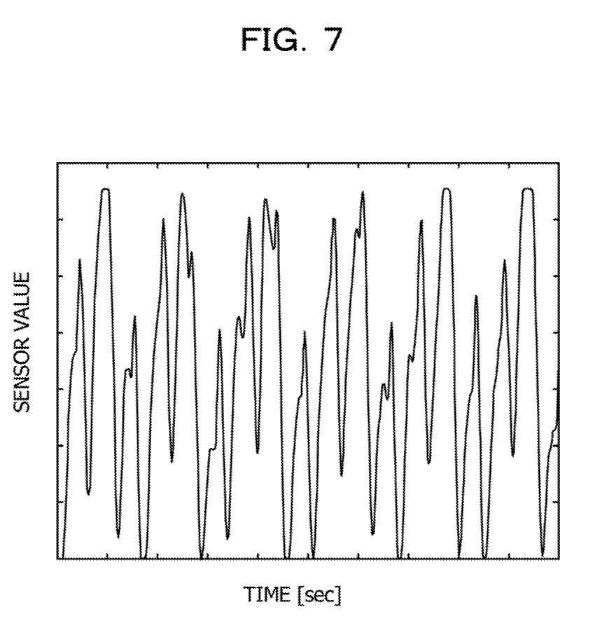

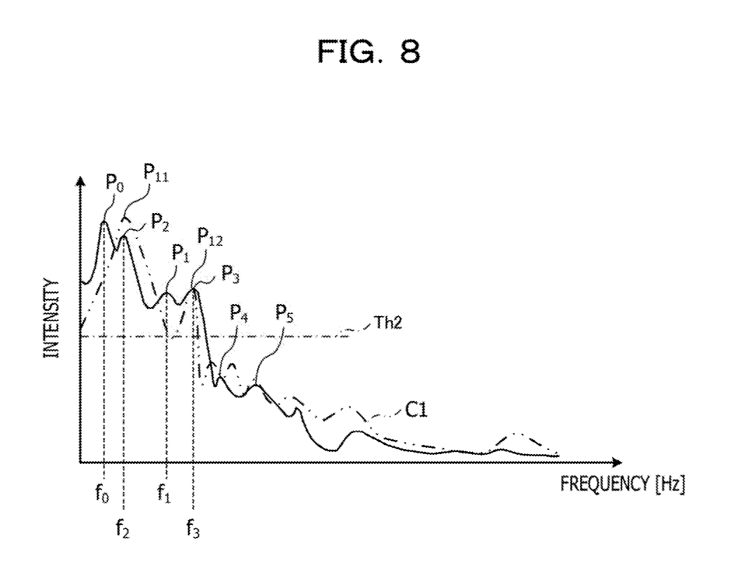

The frequency analysis unit 104 specifies the frequency at which the feature point is obtained based on the chest sensor signal. Specifically, the frequency analysis unit 104 acquires a power spectrum by performing a frequency analysis for the chest sensor signal obtained from the sensor signal acquisition unit 102. FIG. 7 illustrates an example of the chest sensor signal. In FIG. 7, a horizontal axis represents a time and a vertical axis represents a sensor value. Similarly, the frequency analysis is, for example, a FFT and, for example, an analysis result illustrated in FIG. 8 is obtained. FIG. 8 is a graph illustrating an example of the analysis result obtained by the frequency analysis for the chest sensor signal of FIG. 7. In FIG. 8, a horizontal axis represents a frequency and a vertical axis represents intensity (power). In FIG. 8, a waveform C1 illustrated in FIG. 6 is indicated by a two-dot chain line for comparison.

The frequency analysis unit 104 specifies a frequency at which peaks having a predetermined threshold Th2 or more are generated from the analysis result as illustrated in FIG. 8 as a frequency (hereinafter, referred to as a “second peak frequency”) at which the feature point is obtained. The predetermined threshold Th2 is a threshold for extracting only a frequency at which a significant feature point is obtained, and is an adaptive value. The predetermined threshold Th2 may be the same as the predetermined threshold Th1. A plurality of second peak frequencies may exist. Hereinafter, a frequency at which a peak having intensity as a result of the frequency analysis is generated as the first peak frequency and the second peak frequency is also referred to as a “peak frequency”.

In FIG. 8, peaks P₀, P₁, P₂, and P₃ are illustrated as examples of peaks of the predetermined threshold Th2 or more. The peak P₀ is a peak due to the breathing (displacement of the body surface or the lung according to the breathing of the occupant 5), the peak P₁ is a peak due to the heartbeat (displacement of the body surface or organs including the heart according to the heart beat of the occupant 5). Other peaks P₂ and P₃ (same applied to peaks P₄ and P₅ of less than the predetermined threshold Th2) are peaks due to the noise component. For example, the peaks P₂ and P₃ are the feature points generated due to the movement (movement of the chest) of the occupant 5 with respect to the seat 90. The movement of the occupant 5 with respect to the seat 90 causing such peaks P₂ and P₃ to be generated is generated due to the movement and vibration of the vehicle itself, the movement of the occupant 5 himself or herself, or the like. In the example illustrated in FIG. 8, for example, the frequency analysis unit 104 specifies each frequency related to the peaks P₀, P₁, P₂, and P₃ as the second peak frequency.

The comparing unit 107 compares the first peak frequency specified by the frequency analysis unit 103 with the second peak frequency specified by the frequency analysis unit 104. The comparing unit 107 extracts the second peak frequency related to the peak P₀ due to the breathing and the second peak frequency related to the peak P₁ due to the heartbeat from the plurality of the second peak frequencies. Hereinafter, the second peak frequency related to the peak P₀ due to the breathing is also referred to as a “frequency of the breathing” and the second peak frequency related to the peak P₁ due to the heartbeat is also referred to as a “frequency of the heartbeat”. For example, the comparing unit 107 specifies (extracts) the frequency of the breathing and the frequency of the heartbeat included in the second peak frequency based on a comparison result between the first peak frequency specified by the frequency analysis unit 103 and the second peak frequency specified by the frequency analysis unit 104.

Specifically, the comparing unit 107 extracts the second peak frequency which is significantly different from the first peak frequency and specifies (extracts) the frequency of the breathing and the frequency of the heartbeat based on the extracted second peak frequency from the plurality of the second peak frequencies. The frequency of the breathing generally falls within a range of 0.1 to 0.3 Hz and the frequency of the heartbeat generally falls within a range of 0.8 to 3 Hz. Therefore, the comparing unit 107 specifies the second peak frequency which falls within the range of 0.1 to 0.3 Hz and is significantly different from the first peak frequency from the plurality of the second peak frequencies as the frequency of the breathing. When there are two or more second peak frequencies which fall within the range of 0.1 to 0.3 Hz and are significantly different from the first peak frequency, the comparing unit 107 may specify the second peak frequency having a harmonic component as the frequency of the breathing. Similarly, the comparing unit 107 specifies the second peak frequency which falls within the range of 0.8 to 3 Hz and is significantly different from the first peak frequency from the plurality of the second peak frequencies as the frequency of the heartbeat. When there are two or more second peak frequencies which fall within the range of 0.8 to 3 Hz and are significantly different from the first peak frequency, the comparing unit 107 may specify the second peak frequency having a harmonic component as the frequency of the heartbeat.

For example, in the examples illustrated in FIGS. 6 and 8, second peak frequencies f₀ and f₁ among respective second peak frequencies f₀, f₁, f₂, and f₃ related to the peaks P₀, P₁, P₂, and P₃ are significantly different from respective first peak frequencies fit and f₁₂ related to the peaks P₁₁ and P₁₂. For example, the second peak frequencies f₂ and f₃ are respectively substantially the same as the first peak frequencies f₁₁ and f₁₂. This is because the second peak frequencies f₂ and f₃ are peak frequencies related to feature points due to the same movement (movement of the occupant 5 with respect to the seat 90) as those of the first peak frequencies f₁₁ and f₁₂. Specifically, the Doppler sensor 70-1 may detect the movement of the occupant 5 with respect to the seat 90, whereas the Doppler sensor 70-2 may detect the movement related to the breathing of the occupant 5 and the movement related to the heartbeat of the occupant 5 in addition to the same movement. Therefore, among three peak frequencies related to the movement of the occupant 5 with respect to the seat 90, the movement related to the breathing, and the movement related to the heartbeat, the second peak frequency includes the same three peak frequencies, whereas the first peak frequency does not include the peak frequency related to the movement of the occupant 5 with respect to the seat 90. Therefore, it is found there is a high possibility that the second peak frequencies different from the first peak frequency from the plurality of the second peak frequencies correspond to the frequency of the breathing and the frequency of the heartbeat. In this way, the comparing unit 107 may specify the frequency of the breathing and the frequency of the heartbeat with high accuracy based on the comparison result between the first peak frequency and the second peak frequency.

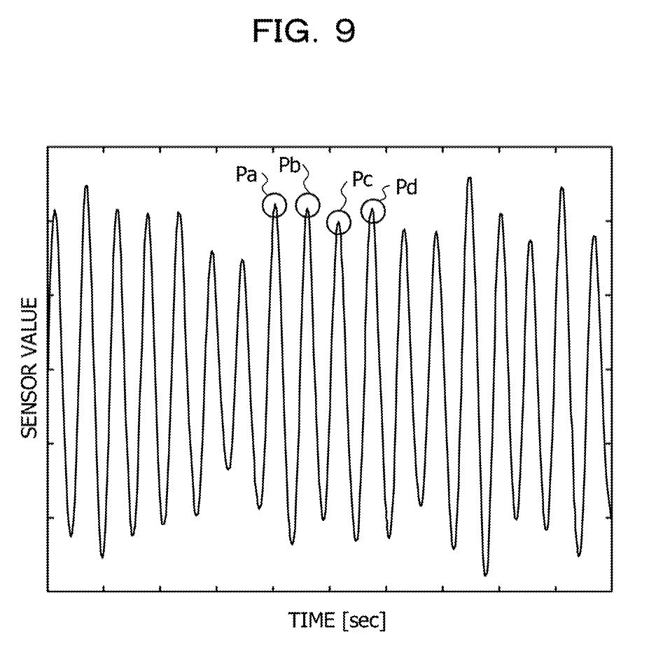

The heartbeat filter processing unit 108 performs filter processing for the chest sensor signal based on the frequency of the heartbeat which is specified by the comparing unit 107. Specifically, a filter processing unit 32 extracts a waveform of a frequency component related to the heartbeat from the chest sensor signal. The filter processing is, for example, band-pass filter (BPF) processing and is performed for extracting the heartbeat which is fluctuated every beat. In this case, the band-pass filter may have, for example, a bandwidth of 0.2 Hz centered on the frequency of the heartbeat. Hereinafter, a signal obtained by performing the filter processing with the band-pass filter centered on the frequency of the heartbeat is referred to as a “heartbeat filtering signal”. FIG. 9 illustrates an example of the heartbeat filtering signal. The heartbeat filtering signal illustrated in FIG. 9 is obtained by performing the filter processing for the chest sensor signal of FIG. 7. In FIG. 9, a horizontal axis represents a time and a vertical axis represents a sensor value.

The heartbeat feature point specifying unit 110 specifies a feature point related to the heartbeat in the heartbeat filtering signal. A specifying method of the feature point related to the heartbeat is arbitrary. For example, the feature point related to the heartbeat appears as a peaks (each peak of Pa to Pd of FIG. 9) in the heartbeat filtering signal. The heartbeat feature point specifying unit 110 specifies each peak in the heartbeat filtering signal as each feature point related to the heartbeat. Each peak in the heartbeat filtering signal may be specified, for example, by performing differentiation processing for the heartbeat filtering signal. The heartbeat feature point specifying unit 110 generates heartbeat information based on information representing a time of each peak in the heartbeat filtering signal. The heartbeat information may be information itself representing the time of each peak in the heartbeat filtering signal, or may be information derived based on the time of each peak in the heartbeat filtering signal. For example, the heartbeat information may be information representing a heartbeat interval. In this case, the heartbeat interval may be calculated as a time interval of each peak in the heartbeat filtering signal.

The breathing filter processing unit 109 performs the filter processing for the chest sensor signal based on the frequency of the breathing specified by the comparing unit 107. Specifically, the filter processing unit 32 extracts the waveform of the frequency component related to the breathing from the chest sensor signal. The filter processing is, for example, the band-pass filter processing. In this case, the band-pass filter may have, for example, a bandwidth of 0.2 Hz centered on the frequency of the breathing. Hereinafter, a signal obtained by performing the filter processing for the chest sensor signal by the band-pass filter centered on the frequency of the breathing is referred to as a “breathing filtering signal”.

The breathing feature point specifying unit 111 specifies the feature point related to the breathing in the breathing filtering signal. A specifying method of the feature point related to the breathing is arbitrary. For example, the feature point related to the breathing appears as a peak in the breathing filtering signal similar to the feature point related to the heartbeat which is described above. The breathing feature point specifying unit 111 specifies each peak in the breathing filtering signal as each feature point related to the breathing. Each peak in the breathing filtering signal may be specified, for example, by the differentiation processing for the breathing filtering signal. The breathing feature point specifying unit 111 generates breathing information based on information representing a time of each peak in the breathing filtering signal. The breathing information may be information itself representing the time of each peak in the breathing filtering signal, or may be information derived based on the time of each peak in the breathing filtering signal.

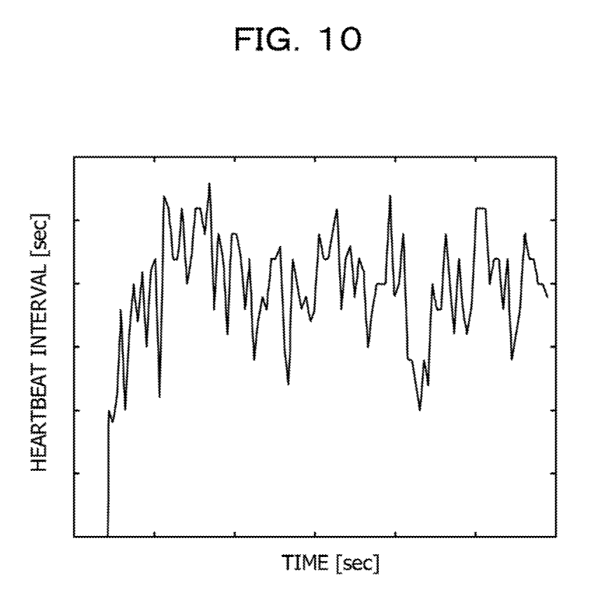

The output unit 112 outputs the heartbeat information and the breathing information obtained by the heartbeat feature point specifying unit 110 and the breathing feature point specifying unit 111, for example, on the display device 22. An output (transmission) destination of the heartbeat information and the breathing information is not limited to the display device 22, but may be, for example, a remotely located monitoring computer (not illustrated) or the like. FIG. 10 illustrates an output example of the heartbeat interval. In FIG. 10, a horizontal axis represents a time and a vertical axis represents the heartbeat interval (unit: second). The output unit 112 may output a result of performing a predetermined frequency analysis on a waveform of the heartbeat interval as illustrated in FIG. 10. The predetermined frequency analysis may be, for example, ae FFT, an autoregressive model (AR model), or the like.

Under an environment in which noise is likely to occur such as in an interior of the vehicle, various noise components caused by the movement and vibration of the vehicle itself, the movement of the occupant 5 himself or herself, or the like are likely to be mixed into the chest sensor signal. Changes (changes excluding the same change due to the heartbeat and the breathing) in a distance between the Doppler sensors 70 and a reflection point on the occupant 5 may all appear as noise components in the chest sensor signal. For example, the noise component that may be mixed into the chest sensor signal is a change in distance between the Doppler sensor 70-2 and the occupant 5, and a change in distance due to disturbance is a main factor. Therefore, under an engagement in which noise is likely to occur, the change in distance due to disturbance occurs in various modes, so that it is difficult to generate the heartbeat information and the breathing information with high accuracy based on only the chest sensor signal.

In this respect, in a state where the occupant 5 is sitting on the seat 90, the movement of the occupant 5 with respect to the seat 90 tends to increase a movement of an upper body. Although the movement of the upper body is different between the waist and the chest, the movement of the thoracic vertebra often accompanies the movement of the lumbar vertebra and moving modes are almost the same. For example, in a case where the upper body of the occupant 5 moves, a chest side moves more largely than a waist side, but the moving modes are almost the same. In the thoracic vertebra and the lumbar vertebra, the intensity (power) related to the noise component of the thoracic vertebra is substantially 3 to 5 times as strong as that of the lumbar vertebra.

More specifically, in a state where the occupant 5 is sitting on the seat 90, as types of the movement of the occupant 5, there are almost no extension (warping behind) or rotation, and increases in bending and side bending. For example, in driving a car, the bending tends to be caused by a change in speed of accelerator, brake, or the like. The side bending is likely to occur by movement to the left and right under an influence of a curve, a step, and an unevenness of a road surface, or the like. In cases of such bending and side bending, the chest side moves more largely than the waist side, but the moving modes are substantially the same.

In a case where the occupant 5 is a driver of a car or the like, the driver unconsciously performs a fine handle operation during driving and the movement becomes a noise component. However, similar to the cases of the bending and the side bending, such movement also has almost the same movement between the waist side and the chest side.

Therefore, it is found that the peak frequency (peak frequency due to the noise component) due to the movement of the occupant 5 with respect to the seat 90 may be specified with high accuracy by comparing the waist sensor signal and the chest sensor signal. For example, the heartbeat information and the breathing information having high accuracy may be generated even in the environment where noise is likely to occur by comparing the waist sensor signal and the chest sensor signal. As described above, according to Example 1, since the heartbeat information and the breathing information are derived based on the waist sensor signal and the chest sensor signal, the heartbeat information and the breathing information may be generated with high accuracy even under the environment where noise is likely to occur.

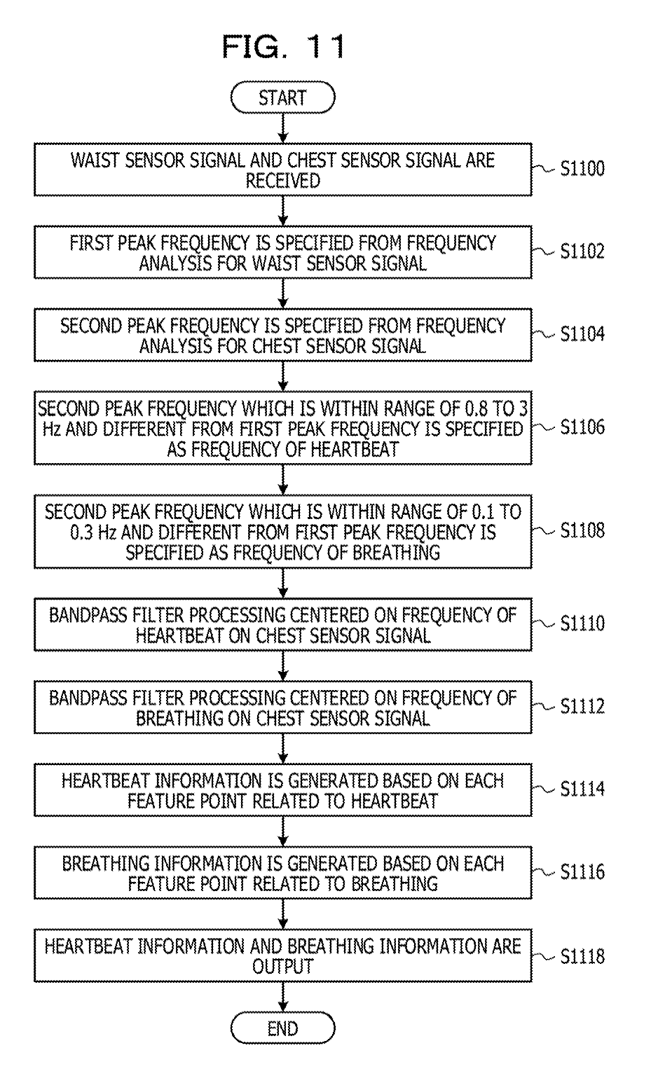

Next, an operation example of the measuring apparatus 10 will be described with reference to FIG. 11.

FIG. 11 is a flowchart illustrating the operation example of the measuring apparatus 10. A process illustrated in FIG. 11 may be executed, for example, for each predetermined period while the measuring apparatus 10 is operated.

In step S1100, the sensor signal acquisition unit 101 and the sensor signal acquisition unit 102 respectively receive the waist sensor signal and the chest sensor signal from the Doppler sensors 70-1 and 70-2. For example, the sensor signal acquisition unit 101 and the sensor signal acquisition unit 102 respectively receive the waist sensor signal and the chest sensor signal of a predetermined period ΔT from before a predetermined time to a present time. The predetermined period ΔT is, for example, 7 seconds.

In step S1102, the frequency analysis unit 103 performs the frequency analysis on the waist sensor signal obtained in step S1100. The frequency analysis unit 103 specifies a first peak frequency as a result of the frequency analysis. A method of specifying the first peak frequency is as described above.

In step S1104, the frequency analysis unit 104 performs the frequency analysis on the chest sensor signal obtained in step S1100. The frequency analysis unit 104 specifies a second peak frequency as a result of the frequency analysis. A method of specifying the second peak frequency is as described above.

In step S1106, the comparing unit 107 specifies the second peak frequency which is within a range of 0.8 to 3 Hz and is significantly different from the first peak frequency obtained in step S1102 from the second peak frequencies obtained in step S1104, as the frequency of the heartbeat.

In step S1108, the comparing unit 107 specifies the second peak frequency which is within a range of 0.1 to 0.3 Hz and is significantly different from the first peak frequency obtained in step S1102 from the second peak frequencies obtained in step S1104, as the frequency of the breathing.

In step S1110, the heartbeat filter processing unit 108 performs the band-pass filter processing for the chest sensor signal obtained in step S1100 centered on the frequency of the heartbeat which is specified in step S1106. Therefore, the heartbeat filtering signal related to the chest sensor signal obtained in step S1100 is obtained.

In step S1112, the breathing filter processing unit 109 performs the band-pass filter processing for the chest sensor signal obtained in step S1100 centered on the frequency of the breathing specified in step S1108. Therefore, the breathing filtering signal related to the chest sensor signal obtained in step S1100 is obtained.

In step S1114, the heartbeat feature point specifying unit 110 specifies each peak in the heartbeat filtering signal obtained in step S1110 as each feature point related to the heartbeat. The heartbeat feature point specifying unit 110 generates the heartbeat information based on each feature point related to the heartbeat.

In step S1116, the breathing feature point specifying unit 111 specifies each peak in the breathing filtering signal obtained in step S1112 as each feature point related to the breathing. The breathing feature point specifying unit 111 generates the breathing information based on each feature point related to the breathing.

In step S1118, the output unit 112 outputs the heartbeat information and the breathing information generated in step S1114 and step S1116.

According to the process illustrated in FIG. 11, for example, the heartbeat information and the breathing information having high accuracy may be output based on the waist sensor signal and the chest sensor signal in real time. Therefore, a state of the occupant in the vehicle may be monitored with high accuracy in real time.

In FIG. 11, the process is executed in a specific order, but the order of the process may be appropriately changed. For example, the order of the process of step S1106 and step S1108 may be reversed, the order of the process of step S1110 and step S1112 may be reversed, or the order of the process of step S1114 and step S1116 may be reversed. For example, step S1106 and step S1110, step S1114 and step S1108, and step S1112 and step S1116 may be executed as one set for each set in this order.

### Example 2

A measuring apparatus 10A according to Example 2 is different from the measuring apparatus 10 according to Example 1 in that an inertial sensor is used in addition to the Doppler sensors 70. In Example 2, the same reference numerals are given to the same configuration elements as those in Example 1 described above.

First, prior to the description of the measuring apparatus 10A according to Example 2, an inertial sensor 60 will be described.

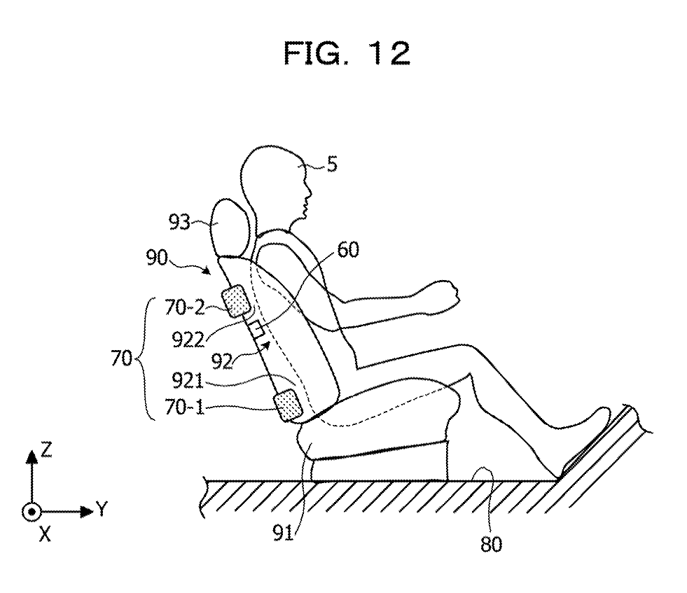

FIG. 12 is a view illustrating an example of a mounting state of the inertial sensor 60. FIG. 12 is provided for explaining a mounting position of the inertial sensor 60 and schematically illustrates the inertial sensor 60 in perspective view.

The inertial sensor 60 is, for example, an acceleration sensor or a gyro sensor. In a case where the inertial sensor 60 is the acceleration sensor, the acceleration sensor detects an acceleration in a direction of each of three axes (X-, Y-, and Z-axes illustrated in FIG. 12) which are orthogonal to each other. In a case where the inertial sensor 60 is the gyro sensor, the gyro sensor detects an angular velocity or an angular acceleration around each of the three axes orthogonal to each other.

The inertial sensor 60 is provided in a backrest 92 of a seat 90. As illustrated in FIG. 12, the inertial sensor 60 may be provided separately from the Doppler sensors 70, or may be built in the Doppler sensors 70. However, the inertial sensor 60 is preferably provided on an upper portion 922 of the backrest 92 as a position where the movement of the seat 90 is easily detected. In this case, the inertial sensor 60 may be built in Doppler sensor 70-2.

In the measuring apparatus 10A according to Example 2, the hardware configuration itself illustrated in FIG. 3 and the radio wave transmitting and receiving unit 25-1 illustrated in FIG. 4 of the measuring apparatus 10 according to Example 1 described above are the same. The measuring apparatus 10A according to Example 2 forms an example of a measuring system by combining with the Doppler sensors 70-1 and 70-2, and the inertial sensor 60.

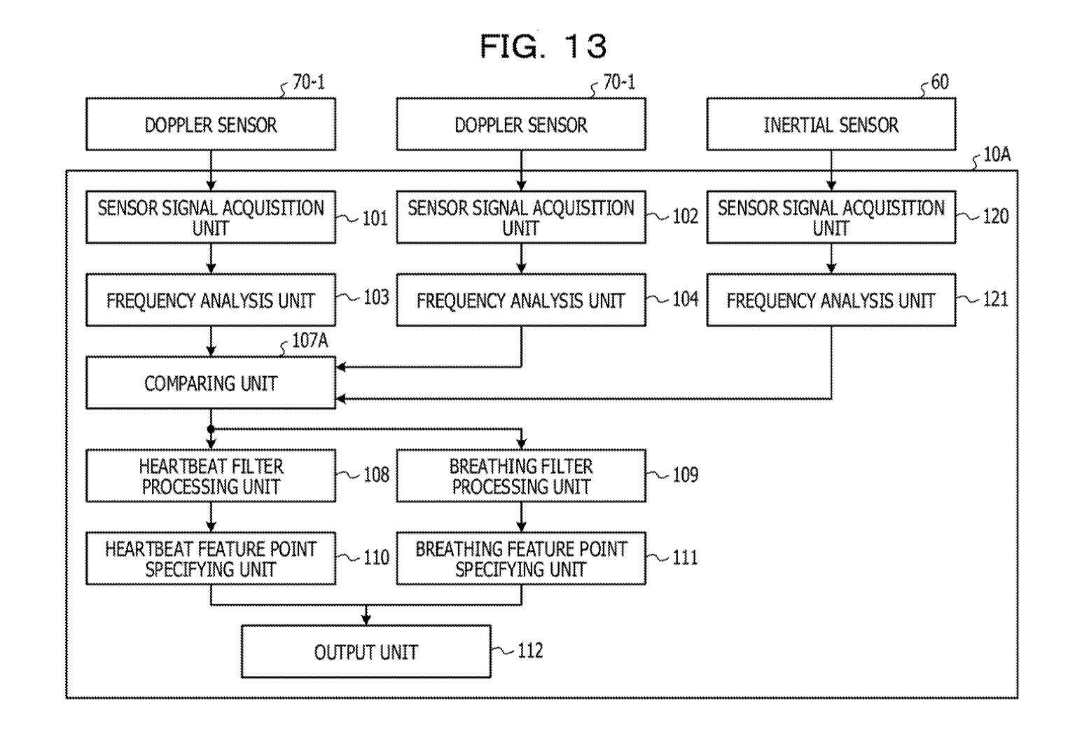

FIG. 13 is a block diagram illustrating an example of a functional configuration of the measuring apparatus 10A according to Example 2. FIG. 13 also illustrates the Doppler sensors 70-1 and 70-2, and the inertial sensor 60. The inertial sensor 60 is connected to the communication interface 17 (see FIG. 3). The measuring apparatus 10A acquires each sensor signal from the Doppler sensors 70-1 and 70-2, and the inertial sensor 60 via the communication interface 17.

The measuring apparatus 10A according to Example 2 is different from the measuring apparatus 10 according to Example 1 described above in that a sensor signal acquisition unit 120 and a frequency analysis unit 121 are added, and the comparing unit 107 is replaced with a comparing unit 107A.

The sensor signal acquisition unit 120 acquires (receives) a sensor signal from the inertial sensor 60. Hereinafter, the sensor signal from the inertial sensor 60 is referred to as an “inertial sensor signal”.

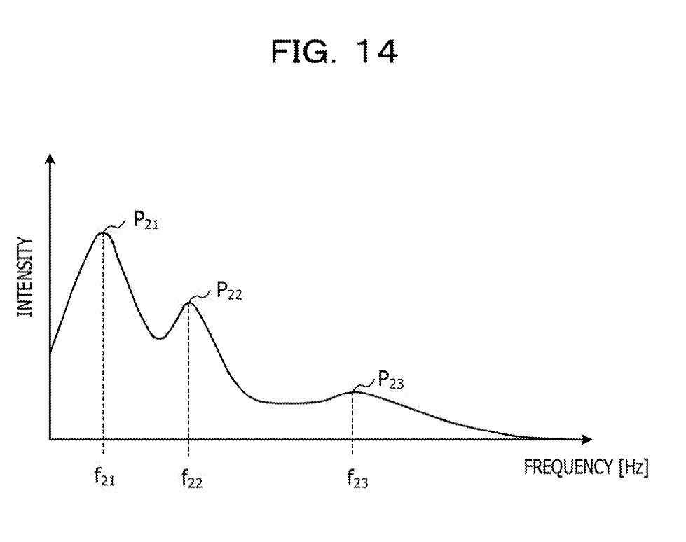

The frequency analysis unit 121 specifies a frequency at which a feature point is obtained based on the inertial sensor signal. Specifically, the frequency analysis unit 121 acquires a power spectrum by performing the frequency analysis for the inertial sensor signal obtained from the sensor signal acquisition unit 120. Similarly, the frequency analysis is an FFT and, for example, an analysis result illustrated in FIG. 14 is obtained. FIG. 14 is a graph illustrating an example of the analysis result obtained by the frequency analysis for the inertial sensor signal. In FIG. 14, a horizontal axis represents a frequency and a vertical axis represents intensity (power).

The frequency analysis unit 121 specifies a frequency, at which peaks of a predetermined threshold Th3 or more from an analysis result as illustrated in FIG. 14 generate, as a frequency (hereinafter, referred to as a “third peak frequency”) at which the feature point is obtained. The predetermined threshold Th3 is a threshold for extracting only a frequency at which a significant feature point is obtained, and is an adaptive value. The predetermined threshold Th3 may be the same as the predetermined threshold Th1. A plurality of third peak frequencies may exist. Peaks P₂₁, P₂₂, and P₂₃ illustrated in FIG. 14 are peaks due to the noise component. For example, the peaks P₂₁, P₂₂, and P₂₃ are the feature points generated due to the movement (vibration or the like) of the seat 90 itself. In the example illustrated in FIG. 14, for example, the frequency analysis unit 121 specifies respective frequencies f₂₁, f₂₂, and f₂₃ related to the peaks P₂₁, P₂₂, and P₂₃, as the third peak frequency.

The frequency analysis unit 121 may specify the third peak frequency with respect to each acceleration signal for the acceleration signal related to each axis included in the inertial sensor signal, or may specify the third peak frequency only using the acceleration signal related to a specific axis. For example, the frequency analysis unit 121 may specify the third peak frequency based on the acceleration signal related to the X-axis and the Z-axis included in the inertial sensor signal. Alternatively, the frequency analysis unit 121 may specify the third peak frequency based on the acceleration signal related to the X-axis or the Z-axis included in the inertial sensor signal. However, the frequency analysis unit 121 preferably specifies the third peak frequency based on the acceleration signal related to at least the Z-axis. This is because vibration in the Z direction is likely to occur in the vehicle.

The comparing unit 107A compares the first peak frequency specified by the frequency analysis unit 103, the second peak frequency specified by the frequency analysis unit 104, and the third peak frequency specified by the frequency analysis unit 121. The comparing unit 107A specifies (extracts) the frequency of the breathing and the frequency of the heartbeat from a plurality of the second peak frequencies. For example, the comparing unit 107A specifies (extracts) the frequency of the breathing and the frequency of the heartbeat based on a comparison result between the first peak frequency, the second peak frequency, and the third peak frequency.

Specifically, the comparing unit 107A extracts the second peak frequency significantly different from any one of the first peak frequency and the third peak frequency from the plurality of the second peak frequencies, and specifies (extracts) the frequency of the breathing and the frequency of the heartbeat based on the extracted second peak frequency. For example, the comparing unit 107A specifies the second peak frequency which is within a range of 0.1 to 0.3 Hz and is significantly different from any one of the first peak frequency and the third peak frequency from the plurality of the second peak frequencies, as the frequency of the breathing. If there are two or more second peak frequencies which are within a range of 0.1 to 0.3 Hz and are significantly different from any one of the first peak frequency and the third peak frequency, the comparing unit 107A may specify the second peak frequency having the harmonic component as the frequency of the breathing. Similarly, the comparing unit 107A specifies the second peak frequency which is within a range of 0.8 to 3 Hz and is significantly different from any one of the first peak frequency and the third peak frequency from the plurality of the second peak frequencies, as the frequency of the heartbeat. If there are two or more second peak frequencies which are within a range of 0.8 to 3 Hz and are significantly different from any one of the first peak frequency and the third peak frequency, the comparing unit 107A may specify the second peak frequency having the harmonic component as the frequency of the heartbeat.

According to Example 2, the same effects as those of Example 1 described above are exerted. For example, according to Example 2, since the heartbeat information and the breathing information are derived based on the waist sensor signal and the chest sensor signal, the heartbeat information and the breathing information may be generated with high accuracy even under the environment where noise is likely to occur.

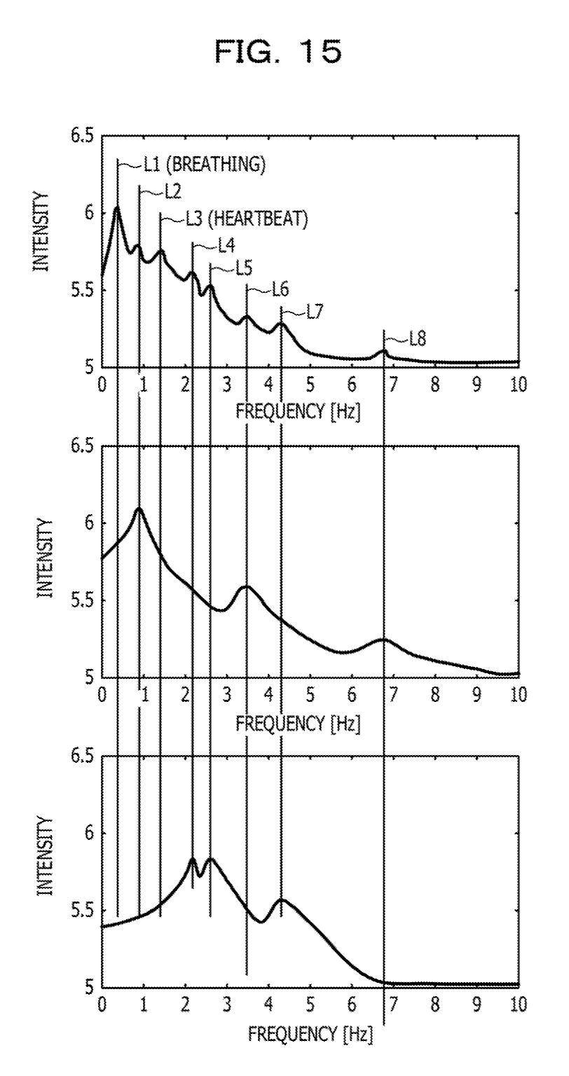

Meanwhile, a noise component caused by the movement (movement of the inertial sensor 60 accordingly) of the seat 90 itself in addition to the movement of the occupant 5 himself or herself with respect to the seat 90 is mixed into the chest sensor signal. For example, when the seat 90 itself vibrates in the Z direction, the Doppler sensor 70-2 itself similarly vibrates, and a position at which the radio waves from the Doppler sensor 70-2 strike the occupant 5 changes at a period according to the vibration. When the position at which the radio waves from the Doppler sensor 70-2 strike the occupant 5 changes, a change in distance between the Doppler sensor 70-2 and the occupant 5 generates due to minute irregularities or the like at a portion of the occupant 5 at the position, and the change in distance may appear as a noise component in the chest sensor signal. Such a noise component may be mixed into the chest sensor signal, but there may be a case where the peak frequency is not generated in the frequency analysis related to the waist sensor signal. This is because the Doppler sensor 70-1 is disposed in a lower portion 921 closer to a support point with respect to the seat surface portion 91 than an upper portion 922 in the backrest 92, so that a displacement due to the vibration of the seat 90 itself or the like is minute. Therefore, it may be difficult to specify the noise component due to the movement of the seat 90 itself based only on comparison between the first peak frequency and the second peak frequency (see FIG. 15 below).

In this respect, since the inertial sensor 60 may detect the movement of the seat 90 itself, the peak frequency related to the noise component due to the movement of the seat 90 itself may be specified from the inertial sensor signal. Therefore, according to Example 2, since the inertial sensor signal is used in addition to the waist sensor signal and the chest sensor signal, even in a case where the noise component due to the movement of the seat 90 itself is mixed, the heartbeat information and the breathing information having high accuracy may be generated.

As described above, the seat 90 includes a damper so as not to directly transmit the vibration to the occupant 5, and the vibration of a main body (for example, a vehicle body or an airframe) of the vehicle and the vibration transmitted to the occupant 5 are different in vibration frequency. Therefore, all the peak frequencies due to the noise component may not be specified only by the third peak frequency based on a frequency analysis result related to the inertial sensor signal from the inertial sensor 60. In this respect, in Example 2, since the waist sensor signal is also used, the peak frequency (peak frequency due to the noise component), which may not be specified by the inertial sensor 60, may be specified and the heartbeat information and the breathing information having high accuracy may be generated.

FIG. 15 is an explanatory view of an effect of Embodiment 2. FIG. illustrates each graph representing a result of the frequency analysis on the chest sensor signal, a result of the frequency analysis on the waist sensor signal, and a result of the frequency analysis on the inertial sensor signal (acceleration signal in the Z-axis direction). In FIG. 15, a horizontal axis represents a frequency and a vertical axis represents intensity (power), and respective graphs have same scales. The result of the frequency analysis illustrated in FIG. 15 represents a result of the frequency analysis related to each sensor signal acquired in a scene different from the chest sensor signal illustrated in FIG. 7.

In FIG. 15, lines L1 to L8 are lines representing respective peak frequencies as a result of the frequency analysis on the chest sensor signal. For the sake of explanation, in FIG. 15, the peaks represented by the lines L1 to L8 also include a peak less than the predetermined threshold Th1 which is described above.

The line L1 among the lines L1 to L8 corresponds to the frequency of the breathing and the line L3 corresponds to the frequency of the heartbeat. Therefore, frequencies related to the line L2 and the lines L4 to L8 among the lines L1 to L8 relate to noise components. As illustrated in FIG. 15, it is found that the frequencies related to the lines L2, L6, and L8 among the line L2 and the lines L4 to L8 may be specified based on the result of the frequency analysis on the waist sensor signal. For example, as illustrated in FIG. 15, others frequencies (for example, frequencies related to the lines L4, 15, and L7) than the frequencies related to the lines L2, 16, and 18 among the line L2 and the lines L4 to L8 may not be specified based on the result of the frequency analysis on the waist sensor signal. On the other hand, the frequencies related to the lines L4, 15, and L7 among the line L2 and the lines L4 to L8 may be specified based on the result of the frequency analysis on the inertial sensor signal. As described above, as illustrated in FIG. 15, it is found that the frequencies (for example, frequencies related to the lines L4, L5, and L7) of the noise component which may not be specified based on the result of the frequency analysis on the waist sensor signal may be specified based on the result of the frequency analysis on the inertial sensor signal. Therefore, it is found that the heartbeat information and the breathing information having further high accuracy may be generated by using the inertial sensor signal.

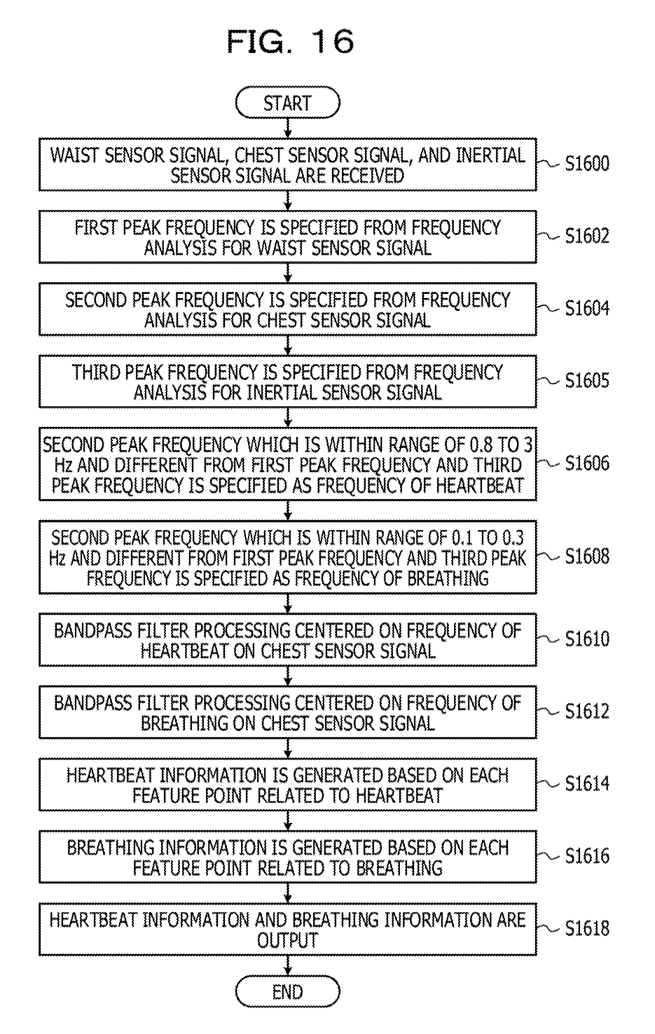

Next, an operation example of the measuring apparatus 10A will be described with reference to FIG. 16.

FIG. 16 is a flowchart illustrating the operation example of the measuring apparatus 10A. The process illustrated in FIG. 16 may be executed for each predetermined period while the measuring apparatus 10A is operated.

In step S1600, the sensor signal acquisition unit 101 and the sensor signal acquisition unit 102 respectively receive the waist sensor signal and the chest sensor signal from the Doppler sensors 70-1 and 70-2. The sensor signal acquisition unit 120 receives the inertial sensor signal from the inertial sensor 60. For example, the sensor signal acquisition unit 101, the sensor signal acquisition unit 102, and the sensor signal acquisition unit 120 respectively receive each sensor signal of the predetermined period ΔT from before a predetermined time to a present time. The predetermined period ΔT is, for example, 7 seconds.

Step S1602 and step S1604 are respectively the same as step S1102 and step S1104 illustrated in FIG. 11, and the description thereof will be omitted.

In step S1605, the frequency analysis unit 121 performs the frequency analysis on the inertial sensor signal obtained in step S1600. The frequency analysis unit 121 specifies the third peak frequency as a result of the frequency analysis. A method of specifying the third peak frequency is as described above.

In step S1606, the comparing unit 107A specifies the frequency of the heartbeat from the second peak frequency obtained in step S1604. Specifically, the comparing unit 107A specifies the second peak frequency which is within a range of 0.8 to 3 Hz and is significantly different from any one of the first peak frequency and the third peak frequency obtained in step S1602 and step S1605, as the frequency of the heartbeat.

In step S1608, the comparing unit 107A specifies the frequency of the breathing from the second peak frequency obtained in step S1604. Specifically, the comparing unit 107A specifies the second peak frequency which is within a range of 0.1 to 0.3 Hz and is significantly different from any one of the first peak frequency and the third peak frequency obtained in step S1602 and step S1605, as the frequency of the breathing.

Step S1610 to step S1618 are respectively the same as step S1110 to step S1118 illustrated in FIG. 11, and the description thereof will be omitted.

According to the process illustrated in FIG. 16, for example, the heartbeat information and the breathing information having high accuracy may be output based on the waist sensor signal, the chest sensor signal, and the inertial sensor signal in real time. Therefore, a state of the occupant in the vehicle may be monitored with high accuracy in real time.

As described in FIG. 11, in FIG. 16, the process is executed in a specific order, but the order of the process may be appropriately changed.

Although each example is described in detail above, it is not limited to a specific example, and various modifications and changes are possible within the scope described in the claims. All or a plurality of configuration elements of the above-mentioned examples may be combined.

For example, in Example 1 (also in Example 2), both the heartbeat information and the breathing information are generated as an example of the biological information, but it is not limited thereto. Only one of the heartbeat information and the breathing information may be generated. For example, in a case where only the heartbeat information is generated, in FIG. 5, the breathing filter processing unit 109 and the breathing feature point specifying unit 111 may be omitted.

In Example 1 (also in Example 2), the heartbeat information and the breathing information are respectively generated based on the breathing filtering signal and the heartbeat filtering signal, but it is not limited thereto. For example, the heartbeat information and the breathing information may be generated without generating the breathing filtering signal and the heartbeat filtering signal based on the frequency of the breathing and the frequency of the heartbeat specified by the comparing unit 107. In this case, for example, when the frequency of the heartbeat which is specified by the comparing unit 107 is f_{heartbeat}, the heartbeat information may represent the frequency f_{heartbeat} itself, or may represent information (heartbeat rate per minute=60×f_{heartbeat}) which may be derived based on the frequency f_{heartbeat}. Similarly, for example, when the frequency of the breathing which is specified by the comparing unit 107 is f_{breathing}, the breathing information may represent the frequency f_{breathing} itself, or may represent information (breathing rate per minute=60×f_{breathing}) which may be derived based on the frequency f_{breathing}.

In Example 1 (also in Example 2), the heartbeat information and the breathing information are respectively generated based on the first peak frequency and the second peak frequency obtained by performing the frequency analysis on the waist sensor signal and the chest sensor signal, but it is not limited thereto. For example, the heartbeat information and the breathing information may be respectively generated based on a differential signal obtained by subtracting the waist sensor signal from the chest sensor signal. In this case, a differential signal S(t) may be generated, for example, as follows based on a waist sensor signal S₁(t) and a chest sensor signal S₂(t).

S₁(t)=Sₙ₁(t)

S₂(t)=S₁₁(t)+S₁₂(t)+Sₙ₂(t)

S(t)=S₂(t)−k×S₁(t)

Here, S₁ₗ(t) and S₁₂(t) respectively represent components related to the heartbeat and the breathing. Sₙ₁(t) and Sₙ₂(t) respectively represent the noise components. k is a coefficient and is an adaptive value, and may be, for example, within a range of 3 to 5. This is because, as described above, in the lumbar vertebra and the thoracic vertebra, the intensity (power) related to the noise component of the thoracic vertebra is substantially 3 to 5 times as strong as that of the lumbar vertebra. Therefore, since the differential signal S(t) is a signal (=S₁₁(t)+S₁₂(t)) in which the noise component in the chest sensor signal S₂(t), the heartbeat information and the breathing information having high accuracy may be generated based on the differential signal S(t). For example, the heartbeat information and the breathing information having high accuracy may be generated based on the peak frequency which is obtained by performing the frequency analysis on the differential signal S(t).

In Example 1 (also in Example 2), the waist sensor signal and the chest sensor signal may be subjected to low-pass filter processing and then the frequency analysis. In this case, a cutoff frequency of the low-pass filter processing is determined according to a search range of the peak frequency, but may be, for example, 5 Hz.

All examples and conditional language provided herein are intended for the pedagogical purposes of aiding the reader in understanding the invention and the concepts contributed by the inventor to further the art, and are not to be construed as limitations to such specifically recited examples and conditions, nor does the organization of such examples in the specification relate to a showing of the superiority and inferiority of the invention. Although one or more embodiments of the present invention have been described in detail, it should be understood that the various changes, substitutions, and alterations could be made hereto without departing from the spirit and scope of the invention.

What is claimed is:

1. A measuring apparatus comprising:
a memory; and
a processor coupled to the memory, the processor being configured to
execute first acquisition processing that includes acquiring a first signal from a first sensor, the first sensor being provided at a first position corresponding to a height of a waist of an occupant in a backrest of a seat on which the occupant sits, the first sensor being configured to detect a movement in accordance with first radio waves,
execute second acquisition processing that includes acquiring a second signal from a second sensor, the second sensor being provided at a second position corresponding to a height of a chest of the occupant in the backrest, the second sensor being configured to detect a movement in accordance with second radio waves,
execute generation processing that includes generating a measurement result related to a predetermined component included in the second signal based on the first signal and the second signal, and
execute output processing that includes outputting the generated measurement result.

2. The measuring apparatus according to claim 1,
wherein the generation processing is configured to generate the measurement result based on a comparison result between a first peak frequency at which a feature point is obtained by performing a frequency analysis for the first signal and a second peak frequency at which a feature point is obtained by performing a frequency analysis for the second signal.

3. The measuring apparatus according to claim 2,
wherein the generation processing is configured to generate the measurement result based on one second peak frequency of a plurality of the second peak frequencies, which is different from the first peak frequency, when the plurality of second peak frequencies exist.

4. The measuring apparatus according to claim 1,
wherein the processor is further configured to execute third acquisition processing that includes acquiring a signal from an inertial sensor provided in the seat, and
wherein the generation processing is configured to generate the measurement result based on a comparison result between a first peak frequency at which a feature point is obtained by performing a frequency analysis for the first signal, a second peak frequency at which a feature point is obtained by performing a frequency analysis for the second signal, and a third peak frequency at which a feature point is obtained by performing a frequency analysis for the signal from the inertial sensor.

5. The measuring apparatus according to claim 4,
wherein the generation processing is further configured to generate the measurement result based on one second peak frequency of a plurality of the second peak frequencies, which is different from both the first peak frequency and the third peak frequency when the plurality of second peak frequencies exist.

6. The measuring apparatus according to claim 4,
wherein the inertial sensor includes an acceleration sensor that detects an acceleration of a component of at least one of a horizontal direction and a vertical direction.

7. The measuring apparatus according to claim 6,
wherein the inertial sensor is provided at the same position as the second position or at a position closer to the second position than to the first position.

8. The measuring apparatus according to claim 3 or 5,
wherein the generation processing is configured to generate the measurement result based on a waveform of a component related to the one second peak frequency obtained by executing filter processing for the second signal.

9. The measuring apparatus according to claim 8,
wherein the filter processing includes band-pass filter processing centered on the one second peak frequency.

10. The measuring apparatus according to claim 8,
wherein the generation processing is configured to generate the measurement result based on an interval of the feature point in the waveform.

11. The measuring apparatus according to claim 3,
wherein the generation processing is configured to generate the measurement result based on the one second peak frequency.

12. The measuring apparatus according to claim 1,
wherein the predetermined component is a component related to heartbeat or breathing of the occupant.

13. The measuring apparatus according to claim 3,
wherein the predetermined component is a component related to heartbeat and breathing of the occupant,
wherein the measurement result includes a first measurement result related to the heartbeat of the occupant and a second measurement result related to the breathing of the occupant, and
wherein the generation processing is configured to generate the first measurement result and the second measurement result based on the second peak frequencies that are different from each other, respectively.

14. The measuring apparatus according to claim 1,
wherein the first sensor includes Doppler sensor configured to transmit the first radio waves in a direction in which the waist of the occupant is positioned and output the first signal based on reflected waves of the first radio waves, and
wherein the second sensor includes Doppler sensor configured to transmit the second radio waves in a direction in which the chest of the occupant is positioned and outputs the second signal based on reflected waves of the second radio waves.

15. The measuring apparatus according to claim 1,
wherein the seat is a seat of a vehicle.

16. A non-transitory computer-readable storage medium for storing a program which causes a processor to perform processing for measuring, the processing comprising:
executing first acquisition processing that includes acquiring a first signal from a first sensor, the first sensor being provided at a first position corresponding to a height of a waist of an occupant in a backrest of a seat on which the occupant sits, the first sensor being configured to detect a movement in accordance with first radio waves;
executing second acquisition processing that includes acquiring a second signal from a second sensor, the second sensor being provided at a second position corresponding to a height of a chest of the occupant in the backrest, the second sensor being configured to detect a movement in accordance with second radio waves;
executing generation processing that includes generating a measurement result related to a predetermined component included in the second signal based on the first signal and the second signal; and
executing output processing that includes outputting the generated measurement result.

17. A measuring system comprising:
a first sensor provided in a backrest of a seat on which an occupant sits, the first sensor being configured to transmit first radio waves in a direction in which a waist of the occupant is positioned, and detect a movement in accordance with the first radio waves;
a second sensor provided in the backrest, the second sensor being configured to transmit second radio waves in a direction in which a chest of the occupant is positioned, and detect a movement in accordance with the second radio waves; and
a processing device configured to output a measurement result related to a predetermined component in accordance with a first signal from the first sensor and a second signal from the second sensor.
